(19) United States
(12) Patent Application Publication
(10) Pub. No.: US 2022/0395499 A1
(43) Pub. Date: Dec. 15, 2022

(54) CONTROLLING EFFECTS AFTER 5HT2A AGONISTS ADMINISTRATION

(71) Applicant: Mind Medicine, Inc., New York, NY (US)

(72) Inventors: Daniel R. KARLIN, New York, NY (US); Robert BARROW, Madison, WI (US)

(73) Assignee: Mind Medicine, Inc., New York, NY (US)

(21) Appl. No.: 17/833,829

(22) Filed: Jun. 6, 2022

Related U.S. Application Data
(60) Provisional application No. 63/210,346, filed on Jun. 14, 2021.

(51) Int. Cl. A61K 31/48; A61P 25/00

(52) U.S. Cl. CPC A61K 31/48; A61P 25/00

(57) ABSTRACT

A composition for treating an individual while reducing acute effects, including effective amounts of a psychedelic drug and a duration shortening agent. A method of treating an individual with a psychedelic drug and reducing or eliminating its acute duration of action, by administering a psychedelic drug to the individual, administering a duration shortening agent to the individual, and shortening and/or reducing and/or eliminating the acute effects of the psychedelic drug. A method of stopping the acute duration of action of a psychedelic drug in an individual, by administering a duration shortening agent to the individual after the individual has taken a psychedelic drug and stopping the acute effects of the psychedelic drug. A method of stopping psychosis due to psychedelic administration, by administering a duration shortening agent to the individual after the individual has taken a psychedelic drug, and stopping psychosis caused by the psychedelic drug.

### BACKGROUND OF THE INVENTION

### 1. Technical Field

The present invention relates to compositions and methods for using 5HT2A antagonists in medical treatments. More specifically, the present invention relates to methods and means for eliminating, shortening or reducing acute effects of 5HT2A agonists.

### 2. Background Art

Lysergic acid diethylamide (LSD) can be used to assist psychotherapy for many indications including anxiety, depression, addiction, personality disorder and others and can also be used to treat other disorders such as cluster headache and migraine and others (Hintzen & Passie, 2010; Liechti, 2017; Nichols, 2016; Passie et al., 2008). LSD targets the 5HT2A receptor, which is a serotonin receptor. Effects of LSD can include altered thoughts, feelings, awareness of surroundings, dilated pupils, increased blood pressure, and increased body temperature.

The duration of action of LSD is long. Doses commonly used in LSD-assisted treatment/psychotherapy are 100-200 μg. A dose of 100 μg produced subjective effects in humans lasting (mean±SD) 8.5±2.0 hours (range: 5.3-12.8 hour) in one representative study (Holze et al., 2019). In other studies, LSD effects similarly lasted 8.2±2.1 hours (range: 5-14 hours) after administration of a 100 μg dose and 11.6±1.7 hours (range: 7-19.5 hours) after administration of a 200 μg dose (Dolder et al., 2017).

The dose-dependent and long duration of action of LSD can be a problem in certain treatment settings. Patients need to be supervised closely and this consumes resources (time, personnel). Additionally, some patients prefer shorter treatments. Further, some patients may also not tolerate the treatment well, in which case a shorter treatment would be needed or a shortening of a treatment that has already started would be needed.

In the past, the problem of the long duration of action of LSD was addressed and partly solved by replacing LSD with shorter-acting substances to assist psychotherapy. In the majority of cases, LSD was replaced by psilocybin which acts for approximately 4-6 hours (Griffiths et al., 2016; Passie et al., 2008) and therefore has a duration of action that is approximately half as long as that of LSD at equivalently psychoactive doses. In part, as a result of the long duration of action of LSD, psilocybin has been used in most of the recent clinical research trials evaluating the efficacy of psychedelics to assess psychotherapy (Carhart-Harris et al., 2017; Carhart-Harris et al., 2016; Griffiths et al., 2016; Grob et al., 2011; Ross et al., 2016). However, a few studies have also used LSD despite its long duration of action (Gasser et al., 2014; Gasser et al., 2015). Additionally, some physicians and patients want to use LSD rather than psilocybin. For example, there is considerably more data on the use and associated safety of LSD than psilocybin. In fact, LSD was mostly used in the 1940's-1970's while psilocybin was only studied more recently mainly after 2000.

U.S. Patent Application Publication No. 20200397752 to Perez Castillo, et al. discloses a combination product for the treatment and/or prevention of psychiatric and/or neurological disorders. The combination product comprises (i) a compound which promotes neurogenesis and has hallucinogenic and/or psychedelic side effects, and (ii) a 5-HT2A receptor antagonist which alleviates and/or removes the hallucinogenic and/or psychedelic side effects caused by the first compound. The 5-HT2A receptor antagonist can be ketanserin. The studies were performed using the hallucinogen dimethyltryptamine (DMT) in animals and to produce neurogenesis and a combination of DMT and ketanserin was used. The invention claims the use of a combination product with exclusively a tryptamine and any 5-HT2A antagonist including ketanserin for use as a medicament. The aim of this treatment is to induce neurogenesis without psychotropic effects.

Pimavanserin is an inverse agonist and antagonist of 5-HT2A receptors, to a lesser extent at 5-HT2C receptors, and has no affinity for 5-HT2B receptors. It is currently approved for treating psychosis (hallucinations and delusions) caused by Parkinson's disease as NUPLAZID® (Acadia Pharmaceuticals). There have also been trials in dementia-related psychosis. The action at the receptors may contribute to its anti-psychotic properties.

Pimavanserin is absorbed in the gastrointestinal tract and is highly bound to plasma protein. Following the administration of a single 34-mg dose of pimavanserin, the time to maximum plasma concentration is six hours, the half-life is approximately 57 hours, and the mean (standard deviation, SD) apparent volume of distribution is 2,173 (307) L. Pimavanserin is metabolized in the liver predominantly by cytochrome P450 (CYP3A4 and CYP3A5) and does not cause significant CYP3A4 inhibition or induction. It has a major active N-desmethylated metabolite AC-279, whose half-life is 200 hours. It does not induce clinically significant antagonism of adrenergic, dopaminergic, histaminergic, or muscarinic receptors and this may be the reason why it does not have movement-related disorders that other antipsychotics have. (Paspe Cruz, M. (2017). Pimavanserin (Nuplazid): a treatment for hallucinations and delusions associated with Parkinson's Disease. P&T 42 (6), 368-371.)

There remains a need for a safe and effective method of using LSD as well as other 5HT2A agonists and methods of eliminating, shortening or aborting the psychedelic experience.

### SUMMARY OF THE INVENTION

The present invention provides for a composition for treating an individual while reducing acute effects, including effective amounts of a psychedelic drug and a duration shortening agent.

The present invention provides for a method of treating an individual with a psychedelic drug and reducing or eliminating its acute duration of action, by administering a psychedelic drug to the individual, administering a duration shortening agent to the individual, and shortening and/or reducing and/or eliminating the acute effects of the psychedelic drug.

The present invention also provides for a method of stopping the acute duration of action of a psychedelic drug in an individual, by administering a duration shortening agent to the individual after the individual has taken a psychedelic drug and stopping the acute effects of the psychedelic drug.

The present invention also provides for a method of stopping psychosis due to psychedelic administration, by administering a duration shortening agent to the individual after the individual has taken a psychedelic drug, and stopping psychosis caused by the psychedelic drug.

The present invention also provides for a method of stopping psychosis in an individual caused by a substance or disease other than Parkinson's disease or schizophrenia, by administering a duration shortening agent to the individual, and stopping psychosis caused by the substance or disease.

### DETAILED DESCRIPTION OF THE INVENTION

The present invention provides generally for short-acting psychedelic treatments for medical conditions. More specifically, the present invention provides for a composition for treating an individual while reducing acute effects, including effective amounts of a psychedelic drug and a duration shortening agent, most preferably pimavanserin. In general, the duration shortening agent reduces the acute effects of the psychedelic drug, and especially reduces or stops psychosis.

The psychedelic drug can be, but is not limited to, 5HT2A agonists such as LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, deuterated forms thereof, analogs thereof, or homologues thereof. Preferably, the dose of the psychedelic is one that provides an effect for at least a few hours (such as at least 2 hours) or has a meaningful effect. A dose of 0.01-1 mg (10-1000 μg can be used of LSD but antagonisms is most meaningful if higher doses of LSD (>0.05 mg) or a psychedelic are used. Psilocybin can be dosed at 1-50 mg, psilocin can be dosed at 1-100 mg, mescaline can be dosed at 10-1000 mg, 5-MeO-DMT can be dosed at 0.2-20 mg, DMT can be dosed at 10-100 mg, DOI can be dosed at 0.1-10 mg, and DOB can be dosed at 0.1-5 mg. Dose ranges can include also very high doses not commonly used clinically.

The duration shortening agent can be any suitable agent that is able to reduce the acute effects of the psychedelic drug and is preferably a 5HT2A receptor antagonist such as pimavanserin, salts thereof, analogs thereof, and homologs thereof. The pimavanserin can be a pimavanserin tartrate salt (as in NUPLAZID®) with the chemical name urea, N-[(4-fluorophenyl)methyl]-N-(1-methyl-4-piperidinyl)-N′-[[4-(2-methylpropoxy)phenyl]methyl]-, (2R,3R)-2,3-dihydroxybutanedioate (2:1). While pimavanserin is referred to herein, it should be understood that any other duration shortening agent can also be used.

Pimavanserin is an antipsychosis agent and is an antagonist of 5HT2A. As described above, the 5HT2A receptor is a serotonin receptor and G protein-coupled receptor that is a target of serotonergic psychedelic drugs like LSD. A dose of 1-100 mg can be used. The duration shortening agent can also be an effect blocking agent.

Most preferably, the compounds are provided separately and administered orally, however, they can also be provided in the same dosage unit and have the same or different release profiles. For example, the dosage unit can be designed to release the psychedelic drug first and subsequently at a later time release the pimavanserin.

The compound of the present invention is administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intramuscular, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating an individual with a psychedelic drug and reducing or eliminating its acute duration of action, by administering a psychedelic drug to the individual, administering a duration shortening agent such as pimavanserin to the individual, and shortening and/or reducing and/or eliminating the acute effects of the psychedelic drug. The psychedelic drug can be administered in amounts of 0.01-1 mg for LSD and pimavanserin can be administered in amounts of 1-100 mg. Pimavanserin can be administered 1 minute to 24 hours after the administration of the psychedelic drug. Any of the psychedelic drugs or duration shortening agents described above can be used in this method. The administration of the duration shortening agent can be at the same time as or at a later time than administration of the psychedelic drug, depending on the formulation. The administration steps can be accomplished by separate oral administration, or as described above, with a single oral dosage unit with release of the psychedelic drug first and subsequent release of the pimavanserin.

The method can also include the step of reducing the time of subjective effects including any drug effect, bad drug effect, anxiety, ego-dissolution, or any other subjective response measure or any other related autonomic response measure (blood pressure, heart rate, or/and pupil size) by 10-100% compared with a treatment consisting of the same amount of the psychedelic drug alone. There can be no recurrence of the psychedelic drug effects after duration shortening agent is administered. In other words, the duration shortening agent remains efficacious in the body of the individual.

The method can be used to reduce time and/or degree of cognitive impairment due to the psychedelic drug, reduce time of treatment session supervision by medical personnel, reduce intensity and/or duration of anxiety or any other acute adverse effects in response to the psychedelic drug, reduce expected acute adverse effects intensity and/or duration due to inadvertent administration of a high dose of the psychedelic drug, reduce expected acute adverse effects intensity and/or duration due to intentional intake of the psychedelic drug (overdose), and reduce expected acute adverse effects duration and/or intensity due to intentional intake of the psychedelic drug in doses considered too high or producing too strong effects after administration.

The present invention also provides for a method of stopping the acute duration of action of a psychedelic drug in an individual, by administering a duration shortening agent such as pimavanserin to the individual after the individual has taken a psychedelic drug and stopping the acute effects of the psychedelic drug. As also described below, this method can be useful in stopping effects of psychedelic drugs that are having an adverse effect on an individual or in the case of an overdose. The duration shortening agent is efficacious in stopping acute effects of the psychedelic when administered after the psychedelic.

The present invention also provides for a method of stopping psychosis due to psychedelic administration, by administering a duration shortening agent such as pimavanserin to the individual after the individual has taken a psychedelic drug, and stopping psychosis caused by the psychedelic drug. The psychosis can cause delusions, hallucinations, talking incoherently, and agitation, and all of these symptoms can be stopped or reduced with the pimavanserin.

The present invention also provides for a method of stopping psychosis in an individual caused by a substance or disease other than Parkinson's disease or schizophrenia, by administering the duration shortening agent to the individual, and stopping psychosis caused by the substance or disease. The disease/condition can be, but is not limited to, bipolar disorder, severe depression, severe stress, severe anxiety, HIV, AIDS, malaria, syphilis, hypoglycemia, lupus, multiple sclerosis, or brain tumors. The substance/medication can be, but is not limited to, cocaine, cannabis, alcohol, muscle relaxants, antihistamines, antidepressants, cardiovascular medications, antihypertensive medications, analgesics, anticonvulsants, anti-Parkinson medications, chemotherapy agents, corticosteroids, or any of the psychedelics described herein.

The invention allows the psychedelic drug experience to be modified (attenuated) with the goal of reducing the acute subjective psychedelic drug effect duration with the goal of 1) reducing time of supervision and 2) avoiding prolonged negative acute treatment effects. For example, the invention targets a reduction of the time of action by 50% to 4-6 hours compared to the classic treatment with LSD alone and reaching a similar duration of action as with psilocybin. The use of a pharmacological antagonist such as ketanserin (40 mg orally) 1 hour prior to the oral administration of LSD at a moderate dose (70-100 μg) has been shown to prevent the LSD experience almost completely (Preller et al., 2017). Administration of ketanserin (40 mg orally) 1 hour prior to a high dose of LSD of 200 μg similarly prevented the LSD experience (Liechti). The present invention uses pimavanserin after administration of the psychedelic drug to shorten the psychedelic drug experience. LSD primarily binds to and activates the serotonin 5HT2A receptor (Rickli et al., 2016) and this receptor interaction is prevented by the 5HT2A receptor antagonist ketanserin which potently binds to this receptor. Researchers have shown strong and unique binding of LSD to the receptor and stated that this process is underlying the long duration of action of LSD in humans (Wacker et al., 2017). Others have shown that LSD acts only as long as it is present in the body and that therefore no special mechanisms at the receptor would be needed to explain its duration of action in humans. Rather the duration is explained well by its pharmacokinetic characteristics (Holze et al., 2019). Importantly, there seems to be significant controversy about whether simple binding of LSD to its target receptor is sufficient to explain its duration of action and therefore it is not obvious that administering a receptor antagonist such as ketanserin would attenuate and shorten the action of LSD in humans. Thus, it is not obvious that the LSD experience can be blocked with a treatment performed after administration based on the known information that ketanserin can prevent an LSD response when ketanserin was administered 1 hour before the LSD.

There are several advantages to the present invention. The action of psychedelic drugs such as LSD that is usually long (8-12 hours) can be made shorter (2-6 hours), allowing shorter and more cost-effective treatment session. In these cases, the duration shortening agent can be administered 1-2 hours after the psychedelic drug to shorten the duration of action by 2-6 hours. The present invention can also attenuate or even stop the psychedelic drug experience using the duration shortening agent to treat patients who a) do not respond well to psychedelic drugs (horror trip), b) consider the experience as too strong, or c) were overdosed. In all these cases, the duration shortening agent can be given immediately after the need to attenuate/antagonize the psychedelic drug effects becomes evident. In extreme cases, the duration shortening agent can be given immediately after the psychedelic drug.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for treating an individual while reducing acute effects, comprising effective amounts of a psychedelic drug and a duration shortening agent.

2. The composition of claim 1, wherein said psychedelic drug is a 5HT2A agonist chosen from the group consisting of LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, deuterated forms thereof, analogs thereof, and homologues thereof.

3. The composition of claim 1, wherein said psychedelic drug is present in an amount that provides an effect for at least 2 hours.

4. The composition of claim 3, wherein said psychedelic drug is present in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

5. The composition of claim 1, wherein said duration shortening agent is a 5HT2A receptor antagonist.

6. The composition of claim 5, wherein said duration shortening agent is chosen from the group consisting of pimavanserin, salts thereof, analogs thereof, and homologs thereof.

7. The composition of claim 6, wherein said pimavanserin is present in an amount of 1-100 mg.

8. The composition of claim 1, wherein said psychedelic drug and duration shortening agent are in dosage units chosen from the group consisting of separate dosage units, in the same dosage unit with the same release profiles, and in the same dosage unit with different release profiles.

9. A method of treating an individual with a psychedelic drug and reducing or eliminating its acute duration of action, including the steps of:
administering a psychedelic drug to the individual;
administering a duration shortening and/or effect blocking agent to the individual; and
shortening and/or reducing and/or eliminating the acute effects of the psychedelic drug.

10. The method of claim 9, wherein the duration shortening agent is administered 1 minute to 24 hours after administering the psychedelic drug.

11. The method of claim 9, wherein the psychedelic drug is a 5HT2A agonist chosen from the group consisting of LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, deuterated forms thereof, analogs thereof, and homologues thereof.

12. The method of claim 9, wherein the psychedelic drug is administered in an amount that provides an effect for at least 2 hours.

13. The method of claim 12, wherein the psychedelic drug is administered in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

14. The method of claim 9, wherein the duration shortening agent is a 5HT2A receptor antagonist.

15. The method of claim 14, wherein the duration shortening agent is chosen from the group consisting of pimavanserin, salts thereof, analogs thereof, and homologs thereof.

16. The method of claim 15, wherein the pimavanserin is administered in an amount of 1-100 mg.

17. The method of claim 9, wherein the psychedelic drug and duration shortening agent are in dosage units chosen from the group consisting of separate dosage units, in the same dosage unit with the same release profiles, and in the same dosage unit with different release profiles.

18. The method of claim 9, further including the step of reducing the time of subjective effects or/and reducing the amount of effects including any drug effect, bad drug effect, anxiety, ego-dissolution, and autonomic response measures by 10-100% compared with a treatment of the same amount of the psychedelic drug alone.

19. The method of claim 9, further providing no recurrence of the psychedelic drug effects after the duration shortening agent is administered.

20. The method of claim 9, further including a step chosen from the group consisting of reducing time and/or degree of cognitive impairment due to the psychedelic drug, reducing time of treatment session supervision by medical personnel, reducing intensity and/or duration of anxiety or any other acute adverse effects in response to the psychedelic drug, reducing expected acute adverse effects intensity and/or duration due to inadvertent administration of a high dose of the psychedelic drug, reducing expected acute adverse effects intensity and/or duration due to intentional intake of the psychedelic drug, and reducing expected acute adverse effects duration and/or intensity due to intentional intake of the psychedelic drug in doses considered too high or producing too strong effects after administration.

21. A method of stopping the acute duration of action of a psychedelic drug in an individual, including the steps of:
administering a duration shortening and/or effect reducing agent to the individual after the individual has taken a psychedelic drug; and
stopping the acute effects of the psychedelic drug.

22. The method of claim 21, wherein the individual is experiencing an adverse effect due to the psychedelic drug.

23. The method of claim 21, wherein the individual has overdosed on the psychedelic drug.

24. The method of claim 21, wherein the duration shortening agent is administered 1 minute to 24 hours after administering the psychedelic drug.

25. The method of claim 21, wherein the psychedelic drug is a 5HT2A agonist chosen from the group consisting of LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, deuterated forms thereof, analogs thereof, and homologues thereof.

26. The method of claim 21, wherein the psychedelic drug is administered in an amount that provides an effect for at least 2 hours.

27. The method of claim 25, wherein the psychedelic drug is administered in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

28. The method of claim 21, wherein the duration shortening agent is a 5HT2A receptor antagonist.

29. The method of claim 28, wherein the duration shortening agent is chosen from the group consisting of pimavanserin, salts thereof, analogs thereof, and homologs thereof.

30. The method of claim 29, wherein the pimavanserin is administered in an amount of 1-100 mg.

31. The method of claim 21, further providing no recurrence of the psychedelic drug effects after the duration shortening agent is administered.

32. A method of stopping psychosis due to psychedelic administration, including the steps of:
administering a duration shortening agent to the individual after the individual has taken a psychedelic drug; and
stopping psychosis caused by the psychedelic drug.

33. The method of claim 32, wherein said stopping step further includes stopping or reducing a symptom chosen from the group consisting of delusions, hallucinations, talking incoherently, and agitation.

34. The method of claim 32, wherein the duration shortening agent is administered 1 minute to 24 hours after administering the psychedelic drug.

35. The method of claim 32, wherein the psychedelic drug is a 5HT2A agonist chosen from the group consisting of LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, deuterated forms thereof, analogs thereof, and homologues thereof.

36. The method of claim 32, wherein the psychedelic drug is administered in an amount that provides an effect for at least 2 hours.

37. The method of claim 35, wherein the psychedelic drug is administered in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

38. The method of claim 32, wherein the duration shortening agent is a 5HT2A receptor antagonist.

39. The method of claim 38, wherein the duration shortening agent is chosen from the group consisting of pimavanserin, salts thereof, analogs thereof, and homologs thereof.

40. The method of claim 39, wherein the pimavanserin is administered in an amount of 1-100 mg.

41. A method of stopping psychosis due to a substance or disease, including the steps of:
administering a duration shortening agent to the individual caused by a substance or disease other than Parkinson's disease or schizophrenia; and
stopping psychosis caused by the substance or disease.

42. The method of claim 41, wherein the disease is chosen from the group consisting of bipolar disorder, severe depression, severe stress, severe anxiety, HIV, AIDS, malaria, syphilis, hypoglycemia, lupus, multiple sclerosis, and brain tumors.

43. The method of claim 41, wherein the substance is chosen from the group consisting of cocaine, cannabis, alcohol, muscle relaxants, antihistamines, antidepressants, cardiovascular medications, antihypertensive medications, analgesics, anticonvulsants, anti-Parkinson medications, chemotherapy agents, corticosteroids, and psychedelics.

44. The method of claim 41, wherein the duration shortening agent is administered 1 minute to 24 hours after administering the psychedelic drug.

45. The method of claim 41, wherein the psychedelic drug is a 5HT2A agonist chosen from the group consisting of LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, deuterated forms thereof, analogs thereof, and homologues thereof.

46. The method of claim 41, wherein the psychedelic drug is administered in an amount that provides an effect for at least 2 hours.

47. The method of claim 45, wherein the psychedelic drug is administered in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

48. The method of claim 41, wherein the duration shortening agent is a 5HT2A receptor antagonist.

49. The method of claim 48, wherein the duration shortening agent is chosen from the group consisting of pimavanserin, salts thereof, analogs thereof, and homologs thereof.

50. The method of claim 49, wherein the pimavanserin is administered in an amount of 1-100 mg.
